(19) United States
(12) Patent Application Publication
(10) Pub. No.: US 2021/0393663 A1
(43) Pub. Date: Dec. 23, 2021

(54) PHARMACEUTICAL FORMULATION CONTAINING ACTIVE METABOLITES OF REMDESIVIR OR ITS ANALOG FOR INHALATION

(71) Applicants: Cai Gu Huang, Shrewsbury, MA (US); Ning He, Shanghai (CN)

(72) Inventors: Cai Gu Huang, Shrewsbury, MA (US); Ning He, Shanghai (CN)

(73) Assignee: Cai Gu Huang, Shrewsbury, MA (US)

(21) Appl. No.: 17/354,702

(22) Filed: Jun. 22, 2021

Related U.S. Application Data
(60) Provisional application No. 63/042,616, filed on Jun. 23, 2020.

(51) Int. Cl. A61K 31/7064; A61K 9/08; A61K 47/40; A61K 47/18; A61K 47/02; A61K 9/00

(52) U.S. Cl. CPC A61K 31/7064; A61K 9/08; A61K 9/0078; A61K 47/186; A61K 47/02; A61K 47/40

(57) ABSTRACT

The present invention relates to a liquid pharmaceutical formulation and a method for administering the pharmaceutical formulation by nebulizing the pharmaceutical formulation with an inhaler. The propellant-free pharmaceutical formulation comprises: (a) an active substance selected from the group consisting of alanine metabolite, nucleoside monophosphate, nucleoside triphosphate, and GS-441524; (b) a solvent; (c) a pharmacologically acceptable solubilizing agent; (d) a pharmacologically acceptable preservative; and (e) a pharmacologically acceptable stabilizer.

### PRIORITY STATEMENT

This application claims the benefit of the filing date of U.S. Provisional Patent Application No. 63/042,616, filed on Jun. 23, 2020, which is incorporated herein by reference in its entirety.

### BACKGROUND OF THE INVENTION

The drug remdesivir hydrolyzes through metabolization to form active metabolites such as alanine metabolite (Ala-met), nucleoside monophosphate and finally nucleoside triphosphate (NTP). The chemical structures of each metabolite is given below:

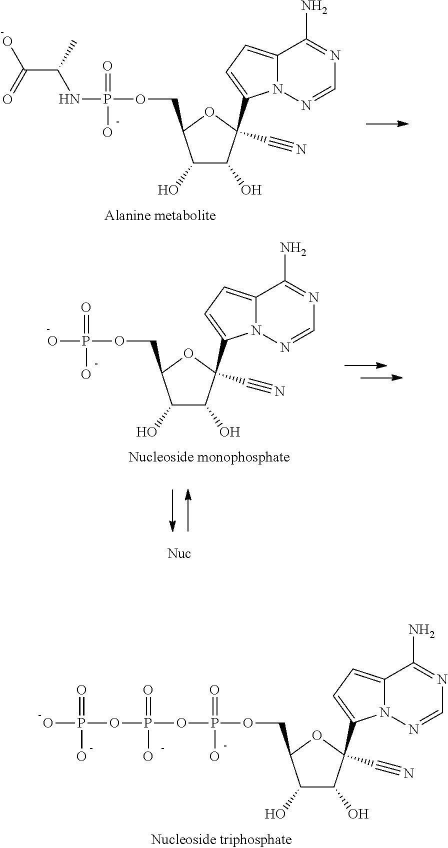 [SMILES: C[C@H](NP(=O)(O)OC[C@H]1O[C@@](C#N)(c2ccc3c(N)ncnn23)[C@H](O)[C@@H]1O)C(=O)O ; N#C[C@@]1(c2ccc3c(N)ncnn23)O[C@H](COP(=O)(O)O)[C@@H](O)[C@H]1O ; N#C[C@@]1(c2ccc3c(N)ncnn23)O[C@H](COP(=O)(O)OP(=O)(O)OP(=O)(O)O)[C@@H](O)[C@H]1O]

A 1-cyano-substituted adenine C-nucleoside ribose analogue (Nuc) exhibits antiviral activity against a number of RNA viruses. The mechanism of action of Nuc requires intracellular anabolism to the active triphosphate metabolite (NTP), which is expected to interfere with the activity of viral RNA-dependent RNA-polymerases (RdRp). Structurally, the 1-cyano group provides potency and selectivity towards viral RNA polymerases, but because of slow first phosphorylation kinetics, modification of parent nucleosides with monophosphate promoieties has the potential to greatly enhance intracellular NTP concentrations. The single S isomer of the 2-ethylbutyl 1-alaninate phosphoramidate prodrug, effectively bypasses the rate-limiting first phosphorylation step of Nuc.

Remdesivir is a pro-drug of its parent adenosine analog, which is metabolized into an active nucleoside triphosphate (NTP) by the host, and currently is under investigation as a broad-spectrum small-molecule antiviral drug that has demonstrated activity against RNA viruses in several families, including Coronaviridae (such as SARSCoV, MERS-CoV, and strains of bat coronaviruses capable of infecting human respiratory epithelial cells), Paramyxoviridae (such as Nipah virus, respiratory syncytial virus, and Hendra virus), and Filoviridae (such as Ebola virus). Remdesivir was originally developed to treat Ebola virus infection.

As a nucleoside analog, remdesivir acts as an RNA-dependent RNA polymerase, targeting the viral genome replication process. The RNA-dependent RNA polymerase is the protein complex that corona viruses (CoVs) use to replicate their RNA-based genomes. After a host metabolizes remdesivir into the active nucleoside triphosphate, the metabolite competes with adenosine triphosphate for incorporation into the nascent RNA strand. The incorporation of this substitute into the new strand results in premature termination of RNA synthesis, halting the growth of the RNA strand after a few more nucleotides are added. Although CoVs have a proof-reading process that is able to detect and remove other nucleoside analogs, rendering them resistant to many of these drugs, the active metabolites of remdesivir appear to outpace this viral proof-reading activity, thus maintaining antiviral activity.

An analog of remdesivir is GS-441524, and its chemical structure is given below:

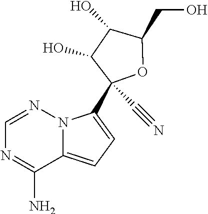 [SMILES: N#C[C@@]1(c2ccc3c(N)ncnn23)O[C@H](CO)[C@@H](O)[C@H]1O]

GS-441524 has antiviral activity against hepatitis C virus, dengue virus, pandemic influenza virus, parainfluenza virus, and SARS coronavirus, and has achieved good results in treating viral infections on cats.

However, remdesivir is currently administered intravenously. Because of the inconvenience of administering a drug intravenously as a solution, as well as the associated side effects due to a long infusion time, it would be preferable to administer remdesivir by inhalation for the treatment of most of the respiratory diseases.

Surprisingly, we found a new approach to more effectively and selectively deliver the active metabolites of remdesivir or its analog (GS-441524) to the lungs and, thus, more effectively inhibit and remove virus from the lungs and other parts of human body. The new delivery method by the soft mist inhalation or nebulization inhalation presents clear and significant clinical benefits, such as availability at the target site, higher efficacy, and less side effects.

Furthermore, administering a formulation of the active metabolites of remdesivir and/or its analog (GS-441524) by inhalation advantageously achieves better distribution of these active agents in the lung, which is especially advantageous when treating or curing a respiratory illness. It is important to increase the lung deposition of a drug delivered by inhalation.

There is a significant need to improve the delivery of the active metabolites of remdesivir and/or its analog (GS-441524) when administered by inhalation so as to increase lung deposition of these active agents. The soft mist or nebulization inhalation device disclosed in US20190030268 can significantly increase the lung deposition of inhalable drugs. These inhalers can nebulize a small amount of a liquid formulation of a drug into an aerosol that is suitable for therapeutic inhalation within a few seconds. Those inhalers are particularly suitable for the inhalation formulations described herein.

In one embodiment, the soft mist or nebulization devices useful for administering the pharmaceutical formulations of the present invention are those in which an amount of less than about 70 microliters of the pharmaceutical formulation can be nebulized in one puff so that the inhalable part of aerosol corresponds to the therapeutically effective quantity. In one embodiment, the soft mist or nebulization devices useful for administering the pharmaceutical formulations of the present invention are those in which an amount of less than about 30 microliters of the pharmaceutical formulation can be nebulized in one puff so that the inhalable part of aerosol corresponds to the therapeutically effective quantity. In one embodiment, the soft mist or nebulization devices useful for administering the pharmaceutical formulations of the present invention are those in which an amount of less than about 15 microliters, or even less, of the pharmaceutical formulation can be nebulized in one puff so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

A mesh based nebulization inhalation device can also significantly increase the lung deposition of inhalable drugs and, thus, is suitable for the inhalation delivery of the pharmaceutical formulations of the invention containing active metabolites of remdesivir and/or its analog (GS-441524).

### SUMMARY OF THE INVENTION

The present invention relates to soft mist or nebulization inhalation containing pharmaceutical formulations of the active metabolites of remdesivir (i.e., alanine metabolite (Ala-met), nucleoside monophosphate, and nucleoside triphosphate (NTP)) and/or its analog (GS-441524) and pharmaceutically acceptable salts or solvates thereof. The pharmaceutical formulations according to the present invention meet high quality standards.

One aspect of the invention is to provide a pharmaceutical soft mist inhalation formulation containing the active metabolites of remdesivir and/or its analog (GS-441524) and other inactive excipients that meets the high standards needed in order to be able to achieve optimum nebulization of the formulation using a soft mist inhaler. In one embodiment, the pharmaceutical formulation is a solution. In one embodiment, the stability of the pharmaceutical formulation is at least one year. In one embodiment, the stability of the pharmaceutical formulation is at least three years. In one embodiment, the stability of the formulation is determined at a temperature ranging from about 15° C. to about 25° C.

Another aspect is to provide formulations that are solutions, which can nebulized using an inhaler device, wherein the produced aerosol falls reproducibly within a specified range for particle size.

Another aspect of the invention is to provide a pharmaceutical nebulization formulation containing the active metabolites of remdesivir and/or its analog (GS-441524) and other inactive excipients that can be administered by nebulization inhalation using an ultra-sonic based or air pressure based nebulizer/inhaler. In one embodiment, the stability of the formulation is at least 1 month. In one embodiment, the stability of the formulation is at least 6 months. In one embodiment, the stability of the formulation is at least one year. In one embodiment, the stability of the formulation is at least three years. In one embodiment, the stability of the formulation is determined at a temperature ranging from about 15° C. to about 25° C.

Another aspect of the invention is to provide stable pharmaceutical formulations that can be administered by soft mist inhalation using atomizer inhalers. In one embodiment, the formulations have substantial long term stability. In one embodiment, the formulations have a storage time of at least about 6 months at a temperature of from about 15° C. to about 25° C. In one embodiment, the formulations have a storage time of at least about 12 months at a temperature of from about 15° C. to about 25° C. In one embodiment, the formulations have a storage time of at least about 24 months at a temperature of from about 15° C. to about 25° C.

Another aspect of the current invention is to provide stable pharmaceutical formulations which can be administered by nebulization inhalation using an ultrasonic, jet, or mesh nebulizer. In one embodiment, the formulations have substantially long-term stability. In one embodiment, the formulations have a storage time of at least about 6 months at a temperature of from about 15° C. to about 25° C. In one embodiment, the formulations have a storage time of at least about 12 months at a temperature of from about 15° C. to about 25° C. In one embodiment, the formulations have a storage time of at least about 24 months at a temperature of from about 15° C. to about 25° C.

In another aspect, the current invention provides a method for treating a viral infection in a patient. In one embodiment, the viral infection is selected from Ebola and Marburg virus (Filoviridae), coronavirus, and new coronavirus COVID-19.

### BRIEF DESCRIPTION OF THE DRAWINGS

FIG. 1 depicts a longitudinal section through an atomizer in the stressed state.

FIG. 2 depicts a counter element of the atomizer.

### DETAILED DESCRIPTION OF THE INVENTION

Administering a formulation containing a drug by inhalation can achieve better distribution of the drug in the lung. It is very important to increase lung deposition of a drug delivered by inhalation.

There is a need in the art to significantly increase lung deposition when a drug is administered by inhalation. The soft mist or nebulization inhalation device disclosed in US20190030268 can significantly increase the lung deposition of inhalable drugs. These inhalers can nebulize a small amount of a liquid formulation into an aerosol that is suitable for therapeutic inhalation within a few seconds. Those inhalers are particularly suitable for administering the liquid formulations of the invention.

In one embodiment, the soft mist or nebulization devices useful for administering the pharmaceutical formulations of the invention are those in which an amount of less than about 70 microliters of the pharmaceutical formulation can be nebulized in one puff so that the inhalable part of aerosol corresponds to the therapeutically effective quantity. In one embodiment, the soft mist or nebulization devices useful for administering the pharmaceutical formulations of the invention are those in which an amount of less than about 30 microliters of the pharmaceutical formulation can be nebulized in one puff so that the inhalable part of aerosol corresponds to the therapeutically effective quantity. In one embodiment, the soft mist or nebulization devices useful for administering the pharmaceutical formulations of the invention are those in which an amount of less than about 15 microliters of the pharmaceutical formulation can be nebulized in one puff so that the inhalable part of aerosol corresponds to the therapeutically effective quantity. In one embodiment, the average particle size of aerosol formed from one puff is less than about 15 microns. In one embodiment, the average particle size of aerosol formed from one puff is less than about 10 microns.

In one embodiment, the nebulization devices useful for administering the pharmaceutical formulations of the invention are those in which an amount of less than about 8 milliliters of pharmaceutical solution can be nebulized in one puff so that the inhalable part of aerosol corresponds to a therapeutically effective quantity. In one embodiment, the nebulization devices useful for administering the pharmaceutical formulations of the invention are those in which an amount of less than about 2 milliliters of pharmaceutical solution can be nebulized in one puff so that the inhalable part of aerosol corresponds to a therapeutically effective quantity. In one embodiment, the nebulization devices useful for administering the pharmaceutical formulations of the invention are those in which an amount of less than about 1 milliliters of pharmaceutical solution can be nebulized in one puff so that the inhalable part of aerosol corresponds to a therapeutically effective quantity. In one embodiment, the average particle size of the aerosol formed from one puff is less than about 15 microns. In one embodiment, the average particle size of the aerosol formed from one puff is less than about 10 microns.

A device of this kind for the propellant-free administration of a metered amount of a liquid pharmaceutical composition for inhalation is described in detail in, for example, US20190030268, entitled “inhalation atomizer comprising a blocking function and a counter”.

The pharmaceutical formulation in the nebulizer is converted into aerosol destined for the lungs. In one embodiment, the pharmaceutical formulation is a solution. The nebulizer uses high pressure to spray the pharmaceutical formulation.

The pharmaceutical formulation is stored in a reservoir in this kind of inhaler. The formulations must not contain any ingredients which might interact with the inhaler to affect the pharmaceutical quality of the solution or of the aerosol produced. In one embodiment, the pharmaceutical formulations are very stable when stored and can be administered directly.

In one embodiment, the pharmaceutical formulations for use with the inhaler described above contain additives, such as the disodium salt of edetic acid (sodium edetate), to reduce the incidence of spray anomalies and to stabilize the formulation. In one embodiment, the formulations have a minimum concentration of sodium edetate.

One aspect of the invention is to provide a pharmaceutical formulation that meets the high standards necessary to achieve optimum nebulization of the formulation using a soft mist inhaler. In one embodiment, the formulations have a stability of at least one year. In one embodiment, the formulations have a stability of at least three years. In one embodiment, the stability of the formulation is determined at a temperature ranging from about 15° C. to about 25° C.

The formulations according to the current invention include one or more active metabolites of remdesivir (such as alanine metabolite (Ala-met), nucleoside monophosphate, and nucleoside triphosphate (NTP)) and/or an analog of remdesivir (GS-441524) or a pharmaceutically acceptable salt or solvate thereof.

In one embodiment, the formulations of the active metabolites of remdesivir and/or an analog of remdesivir (GS-441524) or a pharmaceutically acceptable salt or solvate thereof are dissolved in a solvent. In one embodiment, the solvent comprises water. In one embodiment, the solvent is water.

Another aspect of the invention is to provide formulations that can be nebulized under pressure using an inhaler, which is preferably a soft mist inhaler device. In one embodiment, the formulations are a solution. In one embodiment, the produced aerosol falls reproducibly within a specified range for particle size.

Another aspect of the invention is to provide a nebulization formulation comprising one or more active metabolites of remdesivir and/or an analog of remdesivir (GS-441524) or a pharmaceutically acceptable salt or solvate thereof and other inactive excipients which can be administered by nebulization inhalation. In one embodiment, the aerosol containing the active metabolites of remdesivir and/or an analog of remdesivir (GS-441524) or a pharmaceutically acceptable salt or solvate thereof have a mass median aerodynamic diameter ranging from about 1 micron to about 5 microns. This particle size is able to effectively penetrate the lungs on inhalation. One aspect of the invention is to provide a stable nebulization formulation containing one or more active metabolites of remdesivir and/or an analog of remdesivir (GS-441524) or a pharmaceutically acceptable salt or solvate thereof and other excipients that can be administered by nebulization inhalation.

In another aspect, the current invention provides a method of treating a viral infection in a patient. In one embodiment, the viral infection is selected from Ebola and Marburg virus (Filoviridae); coronavirus, new coronavirus COVID-19, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichinde virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); influenza A virus (Orthomyxoviridae); respiratory syncytial virus(Paramyxoviridae, Pneumovirinae, Pneumovirus); vaccinia virus (Poxviridae, Chordopoxvirinae, Orthopoxvirus); herpes simplex virus type 1, herpes simplex virustype 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Semliki Forest virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); human immunodeficiency virus type 1 (Retroviridae, Lentivirus); influenza AB virus (Orthomyxoviridae, the common ‘flu’ virus); respiratory syncytial virus (Paramyxoviridae, Pneumovirinae, Pneumovirus); Hendra virus, Nipah virus(Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); hepatitis delta virus (hepatitis D virus); herpes simplex virus type 1, herpes simplex virus type 2 (Herpesviridae, Alphaherpesvirinae, Simplexvirus); Middle East Respiratory Syndrome (MERS) virus, severe acute respiratory syndrome CoV (SARS-CoV), and human cytomegalovirus (Herpesviridae, Betaherpesvirinae, Cytomegalovirus).

The effective dose of the active pharmaceutical ingredient (i.e., one or more active metabolites of remdesivir and/or an analog of remdesivir (GS-441524) or a pharmaceutically acceptable salt or solvate thereof) against COVID-19 depends on its bioavailability and clinical efficacy. In one embodiment, the effective dose of the active pharmaceutical ingredient against COVID-19 ranges from about 1 mg to about 500 mg. In one embodiment, the effective dose of the active pharmaceutical ingredient against COVID-19 ranges from about 10 mg to about 300 mg. In one embodiment, the effective dose of the active pharmaceutical ingredient against COVID-19 ranges from about 20 to about 100 mg.

The concentration of the active pharmaceutical ingredient in the finished pharmaceutical preparation depends on the desired therapeutic effect. In one embodiment, the concentration of the active pharmaceutical ingredient in the soft mist formulation ranges from about 0.1 g/100 ml (1 mg/ml) to about 50 g/100 ml (500 mg/ml). In one embodiment, the concentration of the active pharmaceutical ingredient in the soft mist formulation ranges from about 1 g/100 ml (10 mg/ml) to about 20 g/100 ml (200 mg/ml). In one embodiment, the concentration of the active pharmaceutical ingredient in the soft mist formulation ranges from about 2 g/100 ml (20 mg/ml) to about 20 g/100 ml (200 mg/ml).

In one embodiment, the soft mist device useful for administering the pharmaceutical formulation of the invention can atomize about 10 to about 15 microliters of the formulation about 10 to about 15 times per use, so that the inhalable part of aerosol corresponds to a therapeutically effective quantity.

In one embodiment, the formulations include an acid or a base as a pH adjusting agent. In one embodiment, the formulations contain hydrochloric acid and/or a salt thereof as a pH adjusting agent.

Other comparable pH adjusting agents can be used in the present invention. Suitable pH adjusting agents include, but are not limited to, citric acid and sodium hydroxide.

The pH can affect the stability of the formulation. In one embodiment, the pH ranges from about 2.0 to about 6.0. In one embodiment, the pH ranges from about 3.0 to about 5.0.

In one embodiment, the formulations according to the invention include a stabilizer or complexing agent. In one embodiment, the stabilizer or complexing agent is edetic acid (EDTA) or one of the known salts thereof, disodium edetate or edetate disodium dihydrate. In one embodiment, the formulation contains edetic acid and/or a salt thereof

Other comparable stabilizers or complexing agents can also be used. Suitable stabilizers or complexing agents include, but are not limited to, citric acid, edetate disodium, and edetate disodium dihydrate.

The phrases “complexing agent” and “stabilizer,” as used herein, means a molecule that is capable of entering into complex bonds. Preferably, these compounds have the effect of complexing cations. In one embodiment, the concentration of the stabilizer or complexing agent ranges from about 1 mg/100 ml to about 500 mg/100 ml. In one embodiment, the concentration of the stabilizer or complexing agent ranges from about 5 mg/100 ml to about 200 mg/100 ml. In one embodiment, the stabilizer or complexing agent is edetate disodium dihydrate in a concentration of about 10 mg/100 ml.

In one embodiment, all the ingredients of the formulation are present in solution.

The term “additive,” as used herein, means any pharmacologically acceptable and therapeutically useful substance which is not an active substance, but can be formulated together with the active substances in a pharmacologically suitable solvent, in order to improve the qualities of the formulation. Preferably, these substances have no pharmacological effects or no appreciable, or at least no undesirable, pharmacological effects in the context of the desired therapy.

Suitable additives that can be included in the formulations include, but are not limited to, other stabilizers; complexing agents; antioxidants; surfactants; preservatives, which prolong the shelf life of the finished pharmaceutical formulation; vitamins; and/or other additives known in the art.

Preservatives can be added to protect the formulation from contamination with pathogenic bacteria. Suitable preservatives include, but are not limited to, benzalkonium chloride, benzoic acid, and sodium benzoate. In one embodiment, the formulations contain benzalkonium chloride as the only preservative. In one embodiment, the amount of preservative ranges from about 2 mg/100 ml to about 300 mg/100 ml. In one embodiment, the preservative is benzalkonium chloride in an amount of about 10 mg/100 ml.

In one embodiment, the formulations according to the invention include a solubility enhancing agent, such as Tween 80 or a cyclodextrin derivative. In one embodiment, the solubility enhancing agent, is a cyclodextrin derivative or a salt thereof. Without wishing to be bound by theory, it is believed that the solubility enhancing agent improves the solubility of the active ingredients and/or other excipients. In one embodiment, the formulation contains sulfobutylether-β-cyclodextrin or a salt thereof.

In one embodiment, the formulations according to the invention are suitable for soft mist inhalation include a solubility enhancing agents. In one embodiment, the solubility enhancing agent is selected from a group consisting of a surfactant and a cyclodextrin. Suitable surfactants include, but are not limited to, polysorbate, for example, polysorbate 20 and polysorbate 80; poloxamer; sodium dodecyl sulfate (SDS); sodium laurel sulfate; sodium octyl glycoside; polyethyl glycol; polypropyl glycol; copolymers, and mixture thereof. Suitable cyclodextrins include, but are not limited to, β-cyclodextrin, hydroxypropyl-cyclodextrin, and sulfobutylether-β-cyclodextrin. In one embodiment, the solubility enhancing agent is present in an amount ranging from about 1 mg/mL to about 40 g/mL.

Another aspect of the invention is to provide stable pharmaceutical soft mist formulations which can be administered by soft mist inhalation using an atomizer inhaler. In one embodiment, the soft mist formulation has substantial long-term stability. In one embodiment, the formulations have a storage time of at least about 6 months at a temperature of from about 15° C. to about 25° C. In one embodiment, the formulations have a storage time of at least about 12 months at a temperature of from about 15° C. to about 25° C. In one embodiment, the formulations have a storage time of at least about 24 months at a temperature of from about 15° C. to about 25° C.

Another aspect of the invention is to provide pharmaceutical formulations of nebulization solutions that can be administered by nebulization inhalation using a mesh based, ultra-sonic based, or air pressure-based nebulizer/inhaler. In one embodiment, the stability of the formulation is a storage time of few months or years. In one embodiment, the formulation has a storage time of at least about 1 month. In one embodiment, the formulation has a storage time of at least about 6 months. In one embodiment, the formulation has a storage time of at least about one year. In one embodiment, the formulation has a storage time of at least about three years. In one embodiment, the stability of the formulation is determined at a temperature ranging from about 15° C. to about 25° C.

In one embodiment, the nebulization formulation contains the active ingredients (i.e., one or more active metabolites of remdesivir and/or an analog of remdesivir (GS-441524) or a pharmaceutically acceptable salt or solvate thereof) in combination with other excipients. In one embodiment, the aerosol droplets containing the active ingredient have a mass median aerodynamic diameter ranging from about 1 micron to about 10 microns. In one embodiment, the aerosol droplets containing the active ingredient have a mass median aerodynamic diameter ranging from about 1 micron to about 5 microns. This particle size allows the aerosol to be deposited effectively in the lungs upon inhalation.

In one embodiment, the formulations include sodium chloride. In one embodiment, the concentration of the sodium chloride ranges from about 0 g/100 ml to about 0.9 g/100 ml.

In one embodiment of the nebulization formulations, the concentration of the active ingredients ranges from about 1 mg/100 ml to about 20 g/100 ml. In one embodiment of the nebulization formulations, the concentration of the active ingredients ranges from about 5 mg/100 ml to about 1 g/100 ml.

In one embodiment, the formulations according to the invention include a solubilizing agent. Suitable solubilizing agents include, but are not limited to, a surfactant and a cyclodextrin. Suitable surfactants include, but are not limited to, polysorbate, for example, polysorbate 20 and, polysorbate 80; poloxamer; tween-80; sodium dodecyl sulfate (SDS); sodium laurel sulfate; sodium octyl glycoside; polyethyl glycol; polypropyl glycol; copolymers; and mixtures thereof. Suitable cyclodextrins include, but are not limited to, β-cyclodextrin, hydroxypropyl-cyclodextrin, and sulfobutylether-β-cyclodextrin. In one embodiment, the solubilizing agent is present in an amount ranging from about 1 mg/mL to about 40 g/mL.

Another aspect of the invention is to provide stable pharmaceutical nebulization formulations that can be administered using a mesh-based nebulization inhalation device. In one embodiment, the formulations have substantial long-term storage stability. In one embodiment, the formulations have a storage time of at least about 6 months at a temperature of from about 15° C. to about 25° C. In one embodiment, the formulations have a storage time of at least about 12 months at a temperature of from about 15° C. to about 25° C. In one embodiment, the formulations have a storage time of at least about 24 months at a temperature of from about 15° C. to about 25° C.

The pH influences the stability of the nebulization formulation and helps maintain the solubility of active pharmaceutical ingredients. In one embodiment, the pH is adjusted to the desired pH by adding an acid, e.g., HCl, or by adding a base, e.g., NaOH.

In one embodiment, the pH value of the nebulization formulation ranges from about 3 to about 5.

The nebulization formulations according to the present invention can be filled into canisters to form a highly stable formulation for use in a nebulization device. The formulations exhibit substantially no particle growth or change of morphology. There is also no, or substantially no, problem of suspended particles being deposited on the surface of either canisters or valves, so that the formulations can be discharged from a suitable nebulization device with high dose uniformity. In one embodiment, the nebulizer is selected from an ultrasonic nebulizer; a jet nebulizer; or a mesh nebulizer, such as Pari eFlow nebulization inhaler; or other commercially available ultrasonic nebulizer, jet nebulizer, or mesh nebulizer.

In one embodiment, the inhalation device is a soft mist inhaler. To produce the aerosols according to the invention, the pharmaceutical formulation is preferably used in an inhaler of the kind described herein. Here again we expressly mention the patent documents described hereinbefore, to which reference is hereby made.

A device of this kind for administration by soft mist inhalation of a metered amount of a liquid pharmaceutical formulation is described in detail in, for example, US20190030268 entitled “inhalation atomizer comprising a blocking function and a counter”.

The pharmaceutical formulation solution in the nebulizer is converted into an aerosol destined for the lungs. The nebulizer uses high pressure to spray the pharmaceutical solution.

The soft mist inhalation device can be carried anywhere by the patient, since it has a cylindrical shape and a handy size of less than about 8 cm to 18 cm long and 2.5 cm to 5 cm wide. The nebulizer sprays a defined volume of the pharmaceutical formulation out through small nozzles at high pressures, so as to produce inhalable aerosols.

The preferred atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, and an inside part 17.

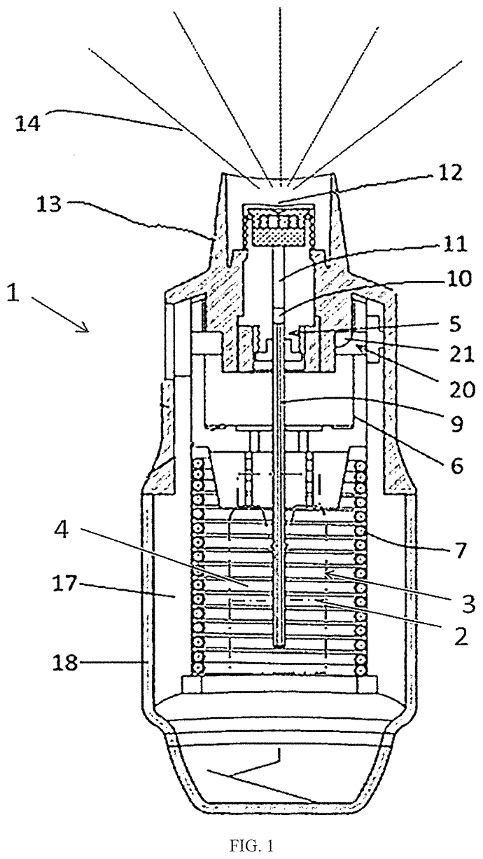

The inhalation atomizer 1 comprising the block function and the counter described above for spraying a medicament fluid 2 is depicted in FIG. 1 in a stressed state. The atomizer 1 comprising the block function and the counter described above is preferred as a portable inhaler and requires no propellant gas.

FIG. 1 shows a longitudinal section through the atomizer in the stressed state.

For the typical atomizer 1 comprising the block function and the counter described above, an aerosol 14 that can be inhaled by a patient is generated through the atomization of the fluid 2, which is preferably formulated as a medicament liquid. The medicament is typically administered at least once a day, more specifically multiple times a day, preferably at predetermined time gaps, according to how seriously the illness affects the patient.

In an embodiment, the atomizer 1 described above has substitutable and insertable vessel 3, which contains the medicament fluid 2. A reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, e.g., up to 200 doses. A typical vessel 3 has a volume of about 2 ml to about 10 ml. A pressure generator 5 in the atomizer 1 is used to deliver and atomize the fluid 2 in a predetermined dosage amount. The fluid 2 can be released and sprayed in individual doses, specifically from about 5 to about 30 microliters.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

In an embodiment, when drive spring 7 is stressed in an axial direction, the delivering tube 9, the vessel 3 along with the holder 6 will be shifted downwards. Then the fluid 2 will be sucked into the pressure room 11 through delivering tube 9 and the non-return valve 10.

In one embodiment, after releasing the holder 6, the stress is eased. During this process, the delivering tube 9 and closed non-return valve 10 are shifted back upward by releasing the drive spring 7. Consequently, the fluid 2 is under pressure in the pressure room 11. The fluid 2 is then pushed through the nozzle 12 and atomized into an aerosol 14 by the pressure. A patient can inhale the aerosol 14 through the mouthpiece 13, while the air is sucked into the mouthpiece 13 through air inlets 15.

The inhalation atomizer 1 described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In one embodiment of the inhalation atomizer 1 described above has a lower shell 18, which carries the inside part 17, and is rotatable relative to the upper shell 16. As a result of rotation and engagement between the upper unit 17 and the holder 6, through a gear 20, the holder 6 is axially moved counter to the force of the drive spring 7, and the drive spring 7 is stressed.

In an embodiment, in the stressed state, the vessel 3 is shifted downwards and reaches a final position, which is demonstrated in FIG. 1. The drive spring 7 is stressed in this final position. Then the holder 6 is clasped. Therefore, the vessel 3 and the delivering tube 9 are prevented from moving upwards so that the drive spring 7 is stopped from easing.

In an embodiment, the atomizing process occurs after releasing the holder 6. The vessel 3, the delivering tube 9 and the holder 6 are shifted back by the drive spring 7 to the beginning position. This is referred to herein as major shifting. When major shifting occurs, the non-return valve 10 is closed and the fluid 2 is under pressure in the pressure room 11 by the delivering tube 9, and then the fluid 2 is pushed out and atomized by the pressure.

In an embodiment, the inhalation atomizer 1 described above may have a clamping function. During the clamping, the vessel 3 preferably performs a lifting shift for the withdrawal of fluid 2 during the atomizing process. The gear 20 has sliding surfaces 21 on the upper shell 16 and/or on the holder 6, which can make holder 6 move axially when the holder 6 is rotated relative to the upper shell 16.

In an embodiment, the holder 6 is not blocked for too long and can perform the major shifting. Therefore, the fluid 2 is pushed out and atomized.

In an embodiment, when holder 6 is in the clamping position, the sliding surfaces 21 move out of engagement. Then the gear 20 releases the holder 6 for the opposite axial shift.

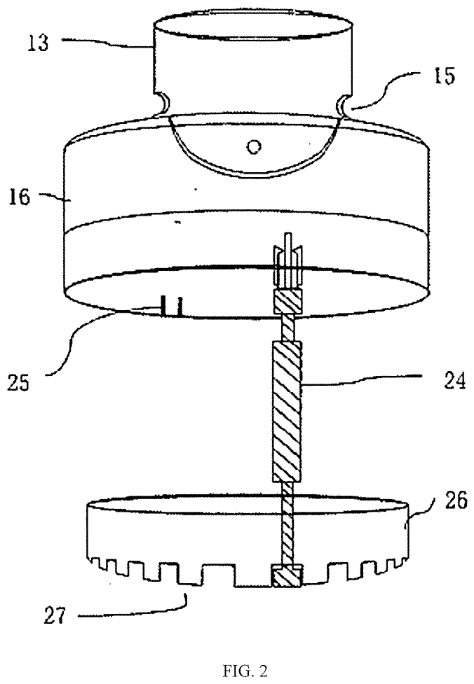

In one embodiment, the atomizer 1 includes a counter element as shown in FIG. 2. The counter element has a worm 24 and a counter ring 26. The counter ring 26 is preferably circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once the predetermined number of actuations is achieved, Protrusion A and Protrusion B will encounter each other and the counter will be prevented from further rotation. Therefore, the atomizer is blocked and stopped from further use. The number of actuations of the device can be counted by the counter.

Atomization devices include, but not limited to, soft mist inhalers, ultrasonic atomizers, air compression atomizers, and mesh based atomizers.

The soft mist inhaler provides pressure to eject a metered dose drug solution. Two high-speed jets are formed, and the two jets collide with each other to form droplets with smaller particles.

With an ultrasonic atomizer, the oscillation signal of the main circuit board is amplified by a high-power triode and transmitted to the ultrasonic wafer. The ultrasonic wafer converts electrical energy into ultrasonic energy. The ultrasonic energy can atomize the water-soluble drug into tiny mist particles ranging from about 1 μm to about 5 μm at normal temperature. With the help of an internal fan, the medicine particles are ejected.

An air compression atomizer is mainly composed of a compressed air source and an atomizer. The compressed gas is suddenly decompressed after passing through the narrow opening at high speed and a negative pressure is generated locally so that the solution of the active substance is sucked out from the container because of a siphon effect. When subject to high-speed air flow, the solution of active substance is broken into small aerosol particles by collision.

Mesh based atomizers contain a stainless steel mesh covered with micropores having a diameter of about 3 μm. The number of micropores exceeds 1,000. The mesh is conical with the cone bottom facing the liquid surface. Under the action of pressure, the vibration frequency of the mesh is about 130 KHz. The high vibration frequency breaks the surface tension of the drug solution contacted with the mesh and produces a low-speed aerosol.

### EXAMPLES

Materials and reagents:

50% benzalkonium chloride aqueous solution purchased from Merck.

Edetate disodium dihydrate purchased from Merck.

Sodium hydroxide purchased from Titan reagents.

Hydrochloric acid purchased from Titan reagents.

Sodium Chloride purchased from Titan reagents

Sulfobutylether-β-cyclodextrin purchased from Zhiyuan Biotechnology.

### Example 1

A nebulization inhalation solution of nucleoside triphosphate (NTP).

The preparation of sample I, sample II, and sample III of a nebulization inhalation solution of nucleoside triphosphate is as follows:

Sodium chloride and nucleoside triphosphate according to the amounts in Table 1 were added to 80 ml of purified water and the resulting mixture sonicated until the components completely dissolved. The solution was adjusted to the target pH with sodium hydroxide or hydrochloric acid. Finally, purified water was added to provide a final volume of 100 ml.

TABLE 1
Components of Sample I, Sample II, Sample III

| Ingredient | Sample I | Sample II | Sample III |
|---|---|---|---|
| Nucleoside | 2,000 mg | 5,000 mg | 10,000 mg |
| Triphosphate | | | |
| Sodium chloride | 900 mg | 600 mg | 300 mg |
| Hydrochloric acid or | To pH 3.0 | To pH 3.5 | To pH 4.0 |
| sodium hydroxide | | | |
| Purified water | Added to | Added to | Added to |
| | 100 ml | 100 ml | 100 ml |

### Example 2

A soft mist inhalation solution of nucleoside triphosphate (NTP).

The preparation of sample IV, sample V, and sample VI of a soft mist inhalation solution of nucleoside triphosphate is as follows:

Edetate disodium dihydrate, 50% benzalkonium chloride aqueous solution, and nucleoside triphosphate according to the amounts in Table 2 were added to 80 ml of purified water and the resulting mixture sonicated until the components completely dissolved. The solution was adjusted to the target pH with sodium hydroxide or hydrochloric acid. Finally, purified water was added to provide a final volume of 100 ml.

TABLE 2
Components of Samples IV, V, and VI

| Ingredients | Sample IV | Sample V | Sample VI |
|---|---|---|---|
| Nucleoside | 10,000 mg | 15,000 mg | 20,000 mg |
| Triphosphate | | | |
| Edetate Disodium | 10 mg | 15 mg | 20 mg |
| Dihydrate | | | |
| 50% benzalkonium | 20 mg | 30 mg | 40 mg |
| chloride aqueous | | | |
| solution | | | |
| Hydrochloric acid or | To pH 3.0 | To pH 3.5 | To pH 4.0 |
| sodium hydroxide | | | |
| Purified water | Added to | Added to | Added to |
| | 100 ml | 100 ml | 100 ml |

### Example 3

A nebulization inhalation solution of alanine metabolite.

The preparation of sample VII, sample VIII, and sample IX of a nebulization inhalation solution of alanine metabolite is as follows:

Sodium chloride and sulfobutylether-β-cyclodextrin according to the amounts in Table 3 were dissolved in 80 ml of purified water. Alanine metabolite according to the amounts in Table 3 was added and the resulting mixture sonicated until the components completely dissolved. The solution was adjusted to the target pH with sodium hydroxide or hydrochloric acid. Finally, purified water was added to final provide a final volume of 100 ml.

TABLE 3
Components of Sample VII, Sample VIII, and Sample IX

| Ingredients | Sample VII | Sample VIII | Sample IX |
|---|---|---|---|
| Alanine metabolite | 500 mg | 750 mg | 1000 mg |
| Sulfobutylether- β - | 5,000 mg | 7,500 mg | 10,000 mg |
| cyclodextrin | | | |
| Sodium chloride | 300 mg | 150 mg | 0 g |
| Hydrochloric acid or | To pH 3.0 | To pH 3.5 | To pH 4.0 |
| sodium hydroxide | | | |
| Purified water | Added to | Added to | Added to |
| | 100 ml | 100 ml | 100 ml |

Example 4

A soft mist inhalation solution of alanine metabolite.

The preparation of sample X, sample XI, and sample XII of a soft mist inhalation solution is as follows:

Sulfobutylether-β-cyclodextrin, edetate disodium dihydrate, and 50% benzalkonium chloride aqueous solution according to the amounts in Table 4 were dissolved in 80 ml of purified water. Alanine metabolite according to the amounts in Table 4 was added and the resulting mixture sonicated until the components completely dissolved. The solution was adjusted to the target pH with sodium hydroxide or hydrochloric acid. Finally, purified water was added to provide a final volume of 100 ml.

TABLE 4
Components of Samples X, XI, and XII

| Ingredient | Sample X | Sample XI | Sample XII |
|---|---|---|---|
| Alanine metabolite | 2,000 g | 2,500 mg | 3,000 mg |
| Sulfobutylether- β - | 5,000 mg | 7,500 mg | 10,000 mg |
| cyclodextrin | | | |
| Edetate Disodium | 10 mg | 15 mg | 20 mg |
| Dihydrate | | | |
| 50% benzalkonium | 20 mg | 30 mg | 40 mg |
| chloride aqueous | | | |
| solution | | | |
| Hydrochloric acid or | To pH 3.0 | To pH 3.5 | To pH 4.0 |
| sodium hydroxide | | | |
| Purified water | Added to | Added to | Added to |
| | 100 ml | 100 ml | 100 ml |

### Example 5

A nebulization inhalation solution of GS-441524.

The preparation of sample XIII, sample XIV, and sample XV of a nebulization inhalation solution of GS-441524 is as follows:

Sodium chloride and sulfobutylether-β-cyclodextrin according to the amounts in Table 5 were dissolved in 80 ml of purified water. GS-441524 according to the amounts in Table 5 was added to the solution and the resulting mixture sonicated until the components completely dissolved. The solution was adjusted to the target pH with sodium hydroxide or hydrochloric acid. Finally, purified water was added to provide a final volume of 100 ml.

TABLE 5
Components of Sample XIII, Sample XIV, and Sample XV

| Ingredient | Sample XIII | Sample XIV | Sample XV |
|---|---|---|---|
| GS-441524 | 500 mg | 750 mg | 1000 mg |
| Sulfobutylether-β- | 5,000 mg | 7,500 mg | 10,000 mg |
| cyclodextrin | | | |
| Sodium chloride | 300 mg | 150 mg | 0 g |
| Hydrochloric acid or | To pH 3.0 | To pH 3.5 | To pH 4.0 |
| sodium hydroxide | | | |
| Purified water | Added to | Added to | Added to |
| | 100 ml | 100 ml | 100 ml |

### Example 6

A soft mist inhalation solution of GS-441524.

The preparation of sample XVI, sample XVII, and sample XVIII of a soft mist inhalation solution is as follows:

Sulfobutylether-β-cyclodextrin, edetate disodium dihydrate, and 50% benzalkonium chloride aqueous solution according to the amounts in Table 6 were dissolved in 80 ml of purified water. GS-441524 according to the amounts in Table 6 was added and the resulting mixture sonicated until the components completely dissolved. The solution was adjusted to the target pH with sodium hydroxide or hydrochloric acid. Finally, purified water was added to provide a final volume of 100 ml.

TABLE 6
Components of Samples XVI, XVII, and XVIII

| Ingredients | Sample XVI | Sample XVII | Sample XVIII |
|---|---|---|---|
| GS-441524 | 2,000 g | 2,500 mg | 3,000 mg |
| Sulfobutylether-β- | 5,000 mg | 7,500 mg | 10,000 mg |
| cyclodextrin | | | |
| Edetate Disodium | 10 mg | 15 mg | 20 mg |
| Dihydrate | | | |
| 50% benzalkonium | 20 mg | 30 mg | 40 mg |
| chloride aqueous | | | |
| solution | | | |
| Hydrochloric acid or | To pH 3.0 | To pH 3.5 | To pH 4.0 |
| sodium hydroxide | | | |
| Purified water | Added to | Added to | Added to |
| | 100 ml | 100 ml | 100 ml |

### Example 7

Aerodynamic Particle Size Distribution:

The aerodynamic particle size distribution of Sample 1 was determined using a Next Generation Pharmaceutical Impactor (NGI).

The preparation of a solution of GS-441524 for administration by soft mist inhalation (sample 1) is as follows:

Sulfobutylether-β-cyclodextrin, edetate disodium dihydrate, and 50% benzalkonium chloride aqueous solution according to the amounts in Table 7 were dissolved in 80 ml of purified water. GS-441524 according to the amounts in Table 7 was added and the resulting mixture sonicated until the components completely dissolved. The solution was adjusted to the target pH with sodium hydroxide or hydrochloric acid. Finally, purified water was added to provide a final volume of 100 ml.

TABLE 7
Components of Sample 1

| Ingredients | Sample1 |
|---|---|
| GS-441524 | 2,000 mg |
| Sulfobutylether-β-cyclodextrin | 5,000 mg |
| Edetate Disodium | 10 mg |
| Dihydrate | |
| 50% benzalkonium chloride | 20 mg |
| aqueous solution | |
| Hydrochloric acid or sodium | To pH 4.0 |
| hydroxide | |
| Purified water | Added to |
| | 100 ml |

The device used to form the aerosol was a soft mist device, the device of this kind for the propellant-free administration of a metered amount of a liquid pharmaceutical composition for inhalation is described in detail in, for example, US20190030268, entitled “inhalation atomizer comprising a blocking function and a counter”. The device was operated at a flow of 30 L/minute and was operated at ambient temperature and a relative humidity (RH) of 90%.

The solution of sample 1 was discharged into the NGI. Fractions of the dose were deposited at different stages of the NGI, in accordance with the particle size of the fraction. Each fraction was washed from the stage and analyzed using HPLC.

The single dose of GS-441524 was 22.1 microliters

The result are shown in Table 8.

TABLE 8
Aerodynamic Particle Size Distribution of Sample 1

| | GS-441524 | | Cut-off |
|---|---|---|---|
| | Dosage | Percentage content | diameters at 15 |
| Deposited | (μg) | at all levels % | L/min (μm) |
| Throat | 83.08 | 19.24 | |
| Stage 1 | 22.6 | 5.23 | 11.72 |
| Stage 2 | 65.16 | 15.09 | 6.4 |
| Stage 3 | 109.12 | 25.27 | 3.99 |
| Stage 4 | 95.08 | 22.02 | 2.3 |
| Stage 5 | 39.32 | 9.11 | 1.36 |
| Stage 6 | 8.6 | 1.99 | 0.83 |
| Stage 7 | 4.48 | 1.04 | 0.54 |
| MOC | 4.36 | 1.01 | 0 |

| | |
|---|---|
| Theoretical dose (μg) | 442 |
| Actual test dose (μg) | 431.08 |
| Recovery rate (%) | 97.69 |
| FPD (μg) | 260.96 |
| FPF (%) | 60.44 |

MOC is Micro-Orifice Collector.
ISM is Impactor Size Mass.
FPF is Fine Particle Fraction.
FPD is fine particle dose.
MMAD is mass median aerodynamic diameter.
GSD is Geometric Standard Deviation.
Stage F is a filter, which is a DDU tube connected to the end of the NGI.

The experimental results in Table 8 show that GS-441524 soft mist inhalation has a very good lung deposition.

### Example 8

### Aerodynamic Particle Size Distribution:

The aerodynamic particle size distribution of Sample 2 was determined using a Next Generation Pharmaceutical Impactor (NGI).

TABLE 9
Components of Sample 2

| Ingredients | Sample 2 |
|---|---|
| GS-441524 | 36 mg |
| Sulfobutylether-β-cyclodextrin | 360 mg |
| NaCl | 21.6 mg |
| Hydrochloric acid or sodium | To pH 4.0 |
| hydroxide | |
| Purified water | Added to |
| | 100 ml |

The preparation of a solution of GS-441524 for administration by nebulization inhalation (sample 2) is as follows:

Sulfobutylether-β-cyclodextrin and NaCl according to the amounts in Table 9 were dissolved in 80 ml of purified water. GS-441524 according to the amount in Table 9 was added and the resulting mixture sonicated until the components completely dissolved. The solution was adjusted to the target pH with sodium hydroxide or hydrochloric acid. Finally, purified water was added to provide a final volume of 100 ml.

The device used to form the aerosol was a PART E-flow device, purchased from PART. The device was held close to the NGI inlet until no aerosol was visible. The flow rate of the NGI was set to 15 L/minute and was operated at ambient temperature and a relative humidity (RH) of 90%.

The solution of sample 2 was discharged into the NGI. Fractions of the dose were deposited at different stages of the NGI, in accordance with the particle size of the fraction. Each fraction was washed from the stage and analyzed using HPLC.

The result are shown in Table 10.

TABLE 10
Aerodynamic Particle Size Distribution of Sample 2

| | GS-441524 | | Cut-off |
|---|---|---|---|
| | Dosage | Percentage content | diameters at 15 |
| Deposited | (μg) | at all levels % | L/min (μm) |
| Throat | 3.32 | 0.92 | |
| Stage 1 | 6.08 | 1.69 | 14.10 |
| Stage 2 | 8.40 | 2.34 | 8.61 |
| Stage 3 | 41.60 | 11.57 | 5.39 |
| Stage 4 | 108.90 | 30.27 | 3.30 |
| Stage 5 | 112.44 | 31.26 | 2.08 |
| Stage 6 | 36.40 | 10.12 | 1.36 |
| Stage 7 | 12.08 | 3.36 | 0.98 |
| MOC | 8.04 | 2.24 | 0 |

| | |
|---|---|
| Theoretical dose (μg) | 360 |
| Actual test dose (μg) | 359.70 |
| Recovery rate (%) | 99.92 |
| FPD ( μg) | 277.86 |
| FPF (%) | 77.25 |

The experimental results in Table 10 show that GS-441524 solution for administration by nebulization inhalation has very good lung deposition

What is claimed is:

1. A liquid, propellant-free pharmaceutical formulation comprising:
(a) one or more active ingredients selected from the group consisting of (i) an active metabolite of remdesiver or a pharmaceutically acceptable salt or solvate thereof and (ii) GS-441524 or a pharmaceutically acceptable salt or solvate thereof;
(b) a solvent;
(c) a pharmacologically acceptable solubilizing agent;
(d) a pharmacologically acceptable preservative; and
(e) a pharmacologically acceptable stabilizer.

2. The pharmaceutical formulation of claim 1, wherein the concentration of the one or more active ingredients ranges from about 0.1 g/100 ml to about 50 g/100 ml.

3. The pharmaceutical formulation of claim 1, wherein the concentration of the one or more active ingredients ranges from about 1 mg/100 ml to about 20 g/100 ml.

3. The pharmaceutical formulation of claim 1, wherein the solvent is water.

4. The pharmaceutical formulation of claim 1, wherein the solvent comprises water.

5. The pharmaceutical formulation of claim 1, wherein the pH of the formulation ranges from about 3.0 to about 5.0.

6. The pharmaceutical formulation of claim 1, wherein the pharmacologically acceptable solubilizing agent is selected from the group consisting of Tween 80 and cyclodextrin derivative or a salt thereof.

7. The pharmaceutical formulation of claim 6, wherein the pharmacologically acceptable solubilizing agent is present in an amount ranging from about 1 mg/100 ml to about 40 g/100 ml.

8. The pharmaceutical formulation of claim 6, wherein the pharmacologically acceptable solubilizing agent is a cyclodextrin derivative or a salt thereof.

9. The pharmaceutical formulation of claim 7, wherein the pharmacologically acceptable solubilizing agent is sulfobutylether-β-cyclodextrin or a salt thereof.

10. The pharmaceutical formulation of claim 1, wherein the preservative is selected from the group consisting of benzalkonium chloride, benzoic acid, and sodium benzoate.

11. The pharmaceutical formulation of claim 1, wherein the preservative is present in an amount ranging from about 2 mg/100 ml to about 300 mg/100 ml.

12. The pharmaceutical formulation of claim 1, wherein the preservative is benzalkonium chloride in an amount of about 10 mg/100 ml.

13. The pharmaceutical formulation of claim 1, wherein the stabilizer is selected from the group consisting of edetic acid (EDTA), disodium edetate, edetate disodium dihydrate, and citric acid.

14. The pharmaceutical formulation of claim 1, wherein the stabilizer is present in an amount ranging from about 1 mg/100 ml to about 500 mg/100 ml.

15. The pharmaceutical formulation of claim 13, wherein stabilizer is edetate disodium dihydrate in a concentration of about 10 mg/100 ml.

16. A method for administering the pharmaceutical formulation of claim 1 comprising nebulizing a defined amount of the pharmaceutical formulation with an inhaler by using pressure to force the pharmaceutical preparation through a nozzle to form an inhalable aerosol.

17. The method of claim 16, wherein the defined amount of the pharmaceutical formulation is less than about 70 microliters.

18. The method of claim 16, wherein the average particle size of the aerosol is less than about 15 microns.

19. The method of claim 16, wherein the aerosol has a mass median aerodynamic diameter ranging from about 1 micron to about 5 microns.

20. The method of claim 16, wherein the pharmaceutical formulation is administered using an inhaler as depicted in FIG. 1.

21. A method of treating asthma or COPD in a patient, comprising administering to the patient the pharmaceutical formulation of claim 1 by inhalation.

22. A liquid, propellant-free pharmaceutical formulation selected from the group consisting of:
(I) an aqueous solution comprising:
(a) nucleoside triphosphate in an amount ranging from about 2,000 mg/100 mL of solution to about 10,000 mg/100 mL of solution; and
(b) sodium chloride in an amount ranging from about 300 mg/100 mL of solution to about 900 mg/100 mL of solution wherein the pH of the formulation ranges from about 3.0 to about 4.0;
(II) an aqueous solution comprising:
(a) nucleoside triphosphate in an amount ranging from about 10,000 mg/100 mL of solution to about 20,000 mg/100 mL of solution;
(b) edetate disodium dihydrate in an amount ranging from about 10 mg/100 mL of solution to about 20 mg/100 mL of solution; and
(c) 50% benzalkonium chloride aqueous solution in an amount ranging from about 20 mg/100 mL of solution to about 40 mg/100 mL of solution;
wherein the pH of the formulation ranges from about 3.0 to about 4.0;
(III) an aqueous solution comprising:
(a) alanine metabolite in an amount ranging from about 5,000 mg/100 mL of solution to about 10,000 mg/100 mL of solution; and
(b) sodium chloride in an amount ranging from about 0 mg/100 mL of solution to about 300 mg/100 mL of solution;
wherein the pH of the formulation ranges from about 3.0 to about 4.0;
(IV) an aqueous solution comprising:
(a) alanine metabolite in an amount ranging from about 2,000 mg/100 mL of solution to about 3,000 mg/100 mL of solution;
(b) edetate disodium dihydrate in an amount ranging from about 10 mg/100 mL of solution to about 20 mg/100 mL of solution; and
(c) 50% benzalkonium chloride aqueous solution in an amount ranging from about 20 mg/100 mL of solution to about 40 mg/100 mL of solution;
wherein the pH of the formulation ranges from about 3.0 to about 4.0;
(V) an aqueous solution comprising:
(a) GS-441524 in an amount ranging from about 500 mg/100 mL of solution to about 1,000 mg/100 mL of solution;
(b) sulfobutylether-β-cyclodextrin in an amount ranging from about 5,000 mg/100 mL of solution to about 10,000 mg/100 mL of solution; and
(c) sodium chloride in an amount ranging from about 0 mg/100 mL of solution to about 300 mg/100 mL of solution;
wherein the pH of the formulation ranges from about 3.0 to about 4.0; and
(VI) an aqueous solution comprising:
(a) GS-441524 in an amount ranging from about 2,000 mg/100 mL of solution to about 3,000 mg/100 mL of solution;
(b) sulfobutylether-β-cyclodextrin in an amount ranging from about 5,000 mg/100 mL of solution to about 10,000 mg/100 mL of solution;
(c) edetate disodium dihydrate in an amount ranging from about 10 mg/100 mL of solution to about 20 mg/100 mL of solution; and
(d) 50% benzalkonium chloride aqueous solution in an amount ranging from about 20 mg/100 mL of solution to about 40 mg/100 mL of solution;
wherein the pH of the formulation ranges from about 3.0 to about 4.0.
